(19) United States
(12) Patent Application Publication
(10) Pub. No.: US 2002/0119204 A1
(43) Pub. Date: Aug. 29, 2002

(54) Composition and method for growing hair

(76) Inventor: Michael Mangan, Oakville (CA)

Correspondence Address: David J. Heller, c/o Ridout & Maybee LLP, Suite 2400, One Queen Street East, Toronto, M5C 3B1, CA

(21) Appl. No.: 10/076,614

(22) Filed: Feb. 19, 2002

Related U.S. Application Data
(60) Provisional application No. 60/268,898, filed on Feb. 16, 2001.

(51) Int. Cl. A61K 35/54

(52) U.S. Cl. 424/582

(57) ABSTRACT

A composition for stimulating hair growth is described comprising an effective amount of mesenchyme from an animal embryo. Methods for orally administering the composition are taught, including maintaining the composition frozen until use, maintaining the composition sublingually prior to swallowing.

### CROSS-REFERENCE

This patent claims priority from U.S. application Ser. No. 60/268,898 filed Feb. 16, 2001, entitled Composition and Method for Growing Hair.

### FIELD OF THE INVENTION

This invention relates to compositions and methods for stimulating growth of hair.

### BACKGROUND OF THE INVENTION

Many preparations for promoting hair growth have been used for prophylaxis or treatment of baldness and thinning of hair.

Preparations for promoting hair growth are generally intended to improve the circulation of the blood in the scalp, attain cleaning, anti-inflammation or sterilization of the scalp, activate enzymes of cells constituting hair follicles and surrounding tissue, improve energy metabolism of hair-matrix cells or depress the action of male sex hormone in scalp. For example, carpronium chloride, vitamin E, an extract of Capsieum annuum L., an extract of Japanese chirata, and a garlic extract have been used to increase the amount of blood stream in hair follicles based on their vasodilative action on peripheral blood vessel, thereby to activate hair follicles. Since baldness is known to be induced by inflammation, anti-inflammatory agents such as glycyrrhizin and allantoin and germicides such as hinokitiol and resorcin have been used for preventing inflammation or bacteria which can induce inflammation.

Vitamins such as vitamin A, vitamin B group, biotin and pantothenic acid derivatives have been used for activating enzymes of hair follicles to promote hair growth. Pentadecanoic acid glyceride has been used for improving the metabolism of hair follicle cells, and female sex hormones such as estradiol and ethynylestradiol have been used for depressing the action of male sex hormone which is believed to be a cause of male pattern baldness.

U.S. Pat. No. 5,702,691 to Ichinose et al. teaches flavanonol derivatives for growing and nourishing hair. U.S. Pat. No. 5,702,691 to Hanada et al. teach purine compounds, pyridylurea compounds, diphenylurea compounds, pyrimidine compounds, imidazole compounds, benzoylaminourea compounds and 4-substituted minopyrrolo [2,3-d]pyrimidine compounds derivatives for growing hair. U.S. Pat. No. 5,508,303 to Isogaya et al. teaches a 5,6,7-trinor-4,8-inter-m-phenylene prostaglandin I₂ derivative for growing hair. Lindner et al (FASEBJ, February 2000, 14(2):319-32) found that recombinantly produced and purified hepatocyte growth factor/scatter factor, when injected intradermally in young mice retards the onset of catagen, a period of controlled regression of the hair follicle. U.S. Pat. No. 6,183,749 to Park teaches a dietary supplement containing herbal extracts and mammalian placenta, which promotes healthy hair growth on the scalp.

However, all of these preparations for promoting hair growth have failed to produce satisfactory hair growth.

Mesenchyme constitutes a morphological term that refers to undifferentiated tissues, from which other tissues and organs of the body originate. The literature refers to some research works involving mesenchyme extracts and specific extra-embryonic membranes extracts in different applications. Some studies have been performed on the healing of burned skin using mesenchyme both in vitro and in vivo (French patent registration no. 2,578,743 to Bontemps; application no. 85/03646). In those studies, mesenchyme preparations improved renewal rate and wound quality.

### SUMMARY OF THE INVENTION

An object of the present invention is to provide a composition for stimulating or promoting hair growth in an animal including a human. Another object of the present invention is to provide a method for stimulating or promoting hair growth of an animal including a human.

The present inventor has found that mesenchyme derivatives from embryonic animals, and in particular bovines, have activities to stimulate or promote hair growth of animals including human when taken orally and/or sublingually.

Thus, the present invention provides a hair-growing composition comprising an extract of mesenchyme from a prenatal animal. The prenatal animal may be an embryo. The prenatal animal may be bovine.

The invention also provides a method of growing hair on an animal, including a human, comprising administering to the animal a composition of the invention in an amount effective for stimulating the hair growth of the animal. The composition may be administered orally. The composition may be administered sublingually. The composition may be administered by maintaining the composition sublingually for a period of time of between 15 seconds and 15 minutes and then subsequently swallowed. The period of time may be 5 minutes.

In an embodiment, prior to administering, the composition is kept frozen. The amount administered may be between 2 ml and 10 ml, preferably two consecutive doses of 3.5 ml each. The composition may be administered twice daily for between 6 and 10 days.

In another embodiment, the method of growing hair further comprises detoxifying the animal by dietary regimen prior to administering the composition. In a further embodiment, topical application of saliva to the animal on a portion of the animal at which hair growth is desired, may be carried out, around the time of administering said composition.

In another embodiment, there is provided a method of treating male erectile dysfunction comprising administering to a male human in need of such treatment an effective amount of a composition of the invention.

### DETAILED DESCRIPTION OF EMBODIMENTS

The mechanisms of how baldness occurs are not yet known in detail. However, the main factors which are at present considered to induce hair loss include:

1. Acatastasia of physiological functions of scalp;

2. Depression of metabolism function in hair follicles and hair bulb;

3. Depression of the function of hair follicles due to male sex hormone action in sebaceous gland, hair follicles and hair root;

4. Mental stress;

5. Genetic factors; and

6. Disease factors.

In addition, aging is believed to accelerate epilation and it is also believed that, as hair follicles age, they suffer depression of cell division ability and cell differentiation ability and depression of metabolic activity, leading to thinning of hair and epilation.

Mesenchyme comprises undifferentiated tissues from which other tissues and organs originate. Mesenchyme liquid extracts of the invention (hereinafter “the extract”) may be prepared from animal embryos. Later gestation fetal material may also be useful. In particular, sheep, cattle and pig embryos are useful, and, in particular, bovine embryos.

The mesenchyme extract of the invention is live frozen and includes stem cells. For bovine products, a licensed veterinarian inspects each animal for health, and each is approved for oral consumption, as part of an active surveillance program for BSE. Microbial, organoleptic and chemical tests are conducted. The raw material then undergoes processing via homogenation, centrifugation, ultrafiltration and asceptic bottling. No heat or chemical solvents are used so that the active molecules maintain their structural integrity. Various commercial laboratories are capable of preparing the extract (Atrium Biotechnologies Inc., Quebec City, Quebec, Canada; Aeterna Laboratories Inc., Quebec, Quebec, Canada)

The extract of the present invention, when taken in the manner and dosages taught herein, is excellent for promoting hair growth, and is effective for any alopecia, and particularly alopecia induced by disease or by toxins, such as chemotherapy.

Since the extract has an unstable chemical structure over time when held at room temperature. Thus, it may be kept frozen until administration to preserve its properties. Dosages are generally 7 ml per day (e.g. two consecutive doses of 3.5 ml each), and may vary between patients due to age, sex, weight and lifestyle. Generally 6-10 applications, and in particular 8 vials (7 ml per vial) are needed for effective treatment.

The dosage is preferably taken on an empty stomach in the morning or evening, either half an hour before or two hours after a meal. A vial containing the extract is held in hand until the material has thawed. The vial is shook, uncapped, and a dosage in the amount of 3.5 ml is poured into the mouth and held under the tongue for is for a period of time of between 15 seconds and 15 minutes and preferably for five minutes before swallowing. A second dose of the same volume is then taken in the same manner. The concentration and volume of the active ingredient in the composition for oral administration is not restricted and may usually be 2 ml to 10 ml. While not wishing to be bound by any particular theory, it appears that while maintaining the extract sublingually, portions of the extract are absorbed in the mouth and/or the saliva's enzymatic catalysts facilitate the absorption and efficacy of the extract.

As an adjunct to the treatment, the application of saliva to the scalp appears to increase efficacy. External topical application of saliva appears to be effective as an enzymatic catalyst and is thus is applied while taking the treatment.

Patients average 10%-55% follicle regrowth after one treatment. The treatment grows hair on entire scalp including hair line. Results are improved if the patient doesn't smoke.

Patients that have maintained their results for a five month period do not lose their newly grown follicles.

From the clinical studies no side effects have been found.

The extract exhibits excellent promotion of hair growth. While not wishing to be bound by any particular theory, the present inventor believes that the extract induces hair growth by providing your body the tissues that produce hair.

The formulation for oral administration may be prepared in the form of tablets, sugar-coated tablets, powder, granules, troches, capsules, balls and syrups.

The present invention thus provides methods for growing hair. As set out in the examples, these methods can include detoxification of the body by dietary regimen prior to taking the extract of the invention. Detoxification prior to taking the extract may facilitate the efficacy of the treatment. The recommended protocol for detoxification is ten to ninety days of herbal and vegetable juicing regime, as prescribed per individual patients needs.

The extracts of the present invention exhibit excellent hair nourishing and hair growing effects. Moreover, live cell therapy has been used for thousands of years for different applications, and thus far there is no record of adverse effects.

The present invention will now be described by way of examples thereof. It should be noted that the examples are presented for the illustration purpose only and should not be interpreted in any restrictive way.

### EXAMPLES

### Example 1

Patient M, a 55 year old male had been completely bald since the age of 45. The patient underwent a detoxification program for 21 days, then underwent treatment with the extract preparation for cellular regrowth. 7 days after commencing treatment with the extract, the patient experienced itching and warmth on his scalp, accompanied by hair growth. The patient also reported feeling greater muscular strength. 20 days later the patient exhibited hair growth in the appearance of a thin crew cut. 30 days later patient M exhibited an approximately 55% increase in hair regrowth evenly across his scalp. 11 months later, the patient has maintained his hair and has suffered no hair loss or side effects.

### Example 2

Patient B, a 71 year old female had hair loss from 30% of her scalp and had worn a wig for 8 years. The patient underwent brain cancer surgery and had skin taken from her hip and grafted to her scalp. Patient B underwent a 21 day detoxification program and the took treatment with the extract of the invention. The patient regained the 30% of hair previously lost and grew thin hair over all of the surgical graft. Patient B no longer wears a wig.

### Example 3

Patient A, a 41 year old male was being treated for prostate problems and had lost hair on 15% of his scalp. Patient A was given a 10 day detoxification program, then commenced taking the extract of the invention. 21 days later, the patient exhibited regrowth of hair on 5% of his scalp. 40 days later the patient exhibited hair regrowth on a further 5%. 90 days later, a further 5% regrowth resulted in reestablishing his previously full head of hair. 11 months later, patient has maintained the new growth.

### Example 4

Patient T, a 38 year old female presented with psoriasis (skin irritation) on her scalp and neck and had 10% hair loss. Patient T underwent a 10 day detoxification program followed by a regime with the extract. 40 days later, the irritation was gone, and the 10% hair loss regained. 11 months later, the patient has had no side effects and no further hair loss.

### Example 5

Patient AO, a 28 year old male had a 25% hair loss from his crown to his frontal region. The patient underwent 8 weeks of detoxification. The patient receive the extract for 2 weeks and in 38 days had hair growth over his crown, representing a regrowth over 12% of his scalp. In 60 days the patient exhibited regrowth to another 10%, primarily the frontal region.

### Example 6

Patient TO, a 43 year old male, underwent a micro graft hair transplant to his crown and frontal areas, but felt it was too painful to endure a second micro graft. Patient declined detoxification treatment and underwent a two week treatment with the extract. Within 10 days the patient exhibited a doubling of hair density.

### Example 7

Patient BC, a 59 year old male had hair loss evenly across 10% of his scalp. The patient also mentioned intense pain in his lower back and feet. He underwent a two week detoxification program and a one week treatment with the extract. 7 days after commencing the extract, patient BC reported that the pain in his feet had subsided and his lower back pain had reduced significantly. At 21 days, there was hair regrowth to 5% of his scalp. At 36 days there was no pain in his back and a further hair regrowth of 10%, resulting in 5% more hair than he had ever had before. 11 months later, the patient has maintained his hair growth with no side effects.

### Example 8

Patient E, a 45 year old male exhibited an overall 10% thinning of his hair. E underwent a 7 day detoxification program and commenced taking the extract from day 8 to day 15. 21 days later patient E exhibited a regrowth of 5%,and at day 50 exhibited full regrowth as the remaining 5% had filled in. Patient E also noticed an increase in muscle strength.

### Example 9

Patient W, a 65 year old male, had 70% hair loss and erectile dysfunction. Patient underwent 3 weeks of detoxification and 2 weeks of treatment with the extract. The patient regrew hair to 5% of his scalp on the crown and also noted a significant improvement with respect to his erectile function.

Other variations and modifications are possible. All such modifications or variations are believed to be within the scope of the invention as defined by the claims appended hereto.

What is claimed is:

1. An composition for growing hair on an animal comprising extract of mesenchyme from a prenatal animal.

2. An composition as claimed in claim 1, wherein said prenatal animal is an embryo.

3. An composition as claimed in either of claim 1 wherein said prenatal animal is bovine.

4. An composition as claimed in either of claim 2 wherein said prenatal animal is bovine.

5. A method of growing hair on an animal, including a human, comprising administering to said animal a composition of claim 1 in an amount effective for stimulating the hair growth of said animal.

6. A method of growing hair on an animal, including a human, comprising administering to said animal a composition of claim 4 in an amount effective for stimulating the hair growth of said animal.

7. A method of growing hair as claimed in claim 5, wherein said composition is administered orally.

8. A method of growing hair as claimed in claim 7, wherein said composition is administered sublingually.

9. A method of growing hair as claimed in claim 8, wherein said composition is administered by maintaining said composition sublingually for a period of time of between 15 seconds and 15 minutes and then subsequently swallowed.

10. A method of growing hair as claimed in claim 9, wherein said period of time is 5 minutes.

11. A method of growing hair as claimed in claim 8, wherein, prior to administering, said composition is kept frozen.

12. A method of growing hair as claimed in claim 8, wherein said composition is administered in an amount of between 2 ml and 10 ml.

13. A method of growing hair as claimed claim 8, wherein said composition is administered twice daily for between 7 and 30 days.

14. A method of growing hair as claimed in claim 8, further comprising detoxifying said animal by dietary regimen prior to administering said composition.

15. A method of growing hair as claimed in claim 8, further comprising topical application of saliva to said animal on a portion of said animal at which hair growth is desired, around the time of administering said composition.

16. A method of growing hair as claimed in claim 8, wherein said animal is human.

17. A method of treating male erectile dysfunction comprising administering to a male human in need of such treatment an effective amount of a composition of claim 1.

18. A method of treating male erectile dysfunction comprising administering to a male human in need of such treatment an effective amount of a composition of claim 3.
